(19) United States
(12) Patent Application Publication
(10) Pub. No.: US 2016/0081874 A1
(43) Pub. Date: Mar. 24, 2016

(54) THERAPEUTIC DEVICE FOR LUNG CANCER METASTASIS

(71) Applicant: Fusheng LI, Shandong (CN)

(72) Inventors: Fusheng LI, Qingdao (CN); An LI, Qingdao (CN); Jing DU, Qingdao (CN); Xiaomei YANG, Qingdao (CN)

(73) Assignee: Fusheng LI, Qingdao, OT (CN)

(21) Appl. No.: 14/890,839

(22) PCT Filed: May 12, 2014
(86) PCT No.: PCT/CN2014/000479

§ 371 Date: Nov. 12, 2015

(30) Foreign Application Priority Data
May 13, 2013 (CN) 201310173374.9
May 13, 2013 (CN) 201320254913.7

(51) Int. Cl. A61H 23/02; A61N 7/02

(52) U.S. Cl. CPC A61H 23/02; A61N 7/02; A61H 2201/0142; A61H 2201/1623

(57) ABSTRACT

A lung cancer metastasis therapeutic equipment consists of a local therapeutic equipment, a bed body of vibration caused by the massotheraphy with injections, an adjustable-speed motor, a motion bar, an oxygen inhaler, and a respiration administration device. The device provides controlling and eliminating the lung cancer metastasis by means of the treatment of the whole generated from the combination of the treatment recovering the function of the respiration system and anti-damage treatment on the structure of the respiration system by means of killing the cancer cells caused by the surgical chemoradiotherapy in the symmetric and equivalent mutual promotion.

### TECHNICAL FIELD

This invention belongs to medical apparatus and instruments, and more specifically, it is the medical equipment integrating massage therapy and medication for treating lung cancer metastasis

### BACKGROUND TECHNOLOGY

Modern medicine hopes to cure cancer through early discovery and early treatment; however, 90% of the patients with advanced lung cancer die from lung cancer metastases and spread. So far, human beings still don't know the cause of the spread of lung cancer, and doctors cannot really cure the lung cancer but to delay the speed of its development once it spreads. Various experts and professors at home and abroad have proposed their suggestions and perspectives in terms of this, which are excerpted as follow:

Patients with non-small cell lung cancer (NSCLC) and with extensive small-cell lung cancer (SCLC) in their late stages (with distant metastasis) cannot be cured, and the chemotherapy can only relieve the symptoms and prolong survival time. Tumor metastasis is a process with multiple steps . . . , and is motivated by the inheritance of cancel cells and its microenvironment as well as the change of its chemical and physical state. More and more evidences show that the immune cells in the tumor microenvironment play a major role in the proliferation process. But we still have little knowledge about the functions of these cells in the lung cancer, so it is in favor of developing new treatment means and finding out new diagnosis and prognosis indicators to better understand their roles in the process of lung cancer metastasis. It is of great promise to adjust the metastatic phenotype through intervening the innate immune response of the host in the future oncotherapy. 40% of the patients with (NSCLC) and nearly 70% of patients with small cell lung cancer belong to hematogenous metastasis lesion in late stages when diagnosing, so the prognosis of the patients with advanced lung cancer is frustrating. In addition, the five-year survival rate is only from 1% to 2%, and the existing of multiple metastases predicts the widespread metastasis which is difficult to cure by the current treatment means. As a result, the main objective for treatment is appeasing and the major means are systemic chemotherapy and molecular targeted drug. Surgical operation, radiotherapy and other treatment means can also be used temporarily to alleviate the symptoms of the metastases. There are clear treatment methods to a small part of patients with oligometastasis NSCLC for targeted therapy, and the clear targeted treatment for patients with isolated metastases can make 15% to 20% of them acquire long term survival. The means of targeted therapy include surgical removal, external radiotherapy, stereotactic radiosurgery (SRS), stereotactic radiotherapy (SRT) and radio frequency ablation (RFA). The strategy and effects of targeted therapy treatment to advanced lung cancer mainly focus on the common metastasis sites of the lung cancer, such as brain, adrenal gland, liver, lung and skeleton. The cancer metastasis severely restricted the therapeutic effect of lung cancer, and the new targeted cancer therapy does not notably improve patients' survival; therefore, new breakthrough should be made to cure lung cancer. Lung Cancer Metastasis (Page 1, 72, 269 and translator page) [America] University of Michigan Venkateshwar Keshamouni, et al. Translated by Song Yong, Shen Xiaokun. People's Medical Publishing House. June 2011, the first edition. Pro. Hai Chunxu in toxicology in the Fourth Military Medical University pointed out that “the discrepancy concentric cell effect should be eliminated as possible in radiotherapy”. The free radical generated by the ionizing radiation during the radiation exposure will damage human body in different degrees, especially the hemopoietic system, which is called “the discrepancy concentric cell effect” and possibly restrict the tumor radiotherapy. So ways should be thought out to eliminate the effect. The risk of cancer patients with radiotherapy to get the cardiovascular and cerebrovascular diseases is obviously higher than patients without radiotherapy. In addition the radiotherapy may damage normal tissues, so that patients have to give up tumor radiotherapy. Medicines that can resist the side effects of radiotherapy should be developed to protect normal tissue cells and kill the tumor cells to the large extent. Health News, 2013.1.2, general news.

Mentality determines outlet and method determines success or failure. “The failure of modern medicine does not lie in its methods but its overall comprehension of the world. Its methods are always silently and unconsciously established on this comprehension, and the failure of modern medicine is because that it lacks a philosophy”. The Healing Power of Illness [Germany] written by Thorwald Dethlefsen and Rüdiger Dahlke, Chunfeng Wenyi Publishing House, September 1999, first edition, page 4. “Since it was proved by facts that there has been no revolutionary progress in the medical technology for cancer in the last fifty years, it is not likely to find out great breakthrough in the foreseeable future. Therefore, it could be possible to change the attack direction toward curing and improving environment”. Qi Lu Evening News, Jun. 18, 2007. Philosophy starts from where science stops, and is going to change the nature of things and directions of therapy. At present, under the circumstance that all the existing technologies will kill part of the cancer cells, it is to equivalently push out a series of physio-chemical technique that can recover human body's metabolism function (including immunity). Both of them depend on each other and equivalently combine together to form an overall synchronization, and treat every pathologic progress and part of cancer rather than local treatment like now, thus generating notable curative effects.

The existing technology Therapeutic Equipment for Cancer Diseases in the series of invention, is ratified by the National Invention Patent ZL200610069987.8,2011.6.29; and for the PCT international patent (PCT/CN2007/003135), it obtained the Japanese invention patent special permission No. 5047301 ratified on Jul. 27, 2012; Therapeutic Equipment for Cancer Spreading and Metastasis national patent ZL201220434659.4 ratified on Mar. 27, 2013. Treatment principles and methods for serious diseases proposed in the patent specification are: to cure serious diseases through symmetrically and equivalently etiological treatment by using the overall medical model based on combining the modern philosophy and science developed from Chinese ancient philosophy as well as the theory of traditional Chinese medicine, which has essential differences from the empiricism mode that cures serious disease through taking structure as the principle thing by using the modern medicine based on modern technology and lacks philosophical theories. The overall factors that combined structure and functions equivalently play the major role rather than local factors, which is the nature of cancer diseases and serious diseases.

Cancer is the general term for various malignant tumors, and the existing technologies Therapeutic Equipment for Cancer Diseases and Therapeutic Equipment for Cancer Spreading and Metastasis expounded the nature of cancer and its spreading and metastasis in details. But they cannot give specific interpretation for the concrete manifestation of the specificities of various malignant tumors. Therefore, the existing technology is short of targeted solutions as to specific therapeutic measures, and mainly focuses on local targeted recovery function in the cancer therapy, while lacking large therapeutic equipment in terms of recovering respiratory function that needs general movements.

### CONTENTS OF THE INVENTION

Since the interaction between the organ structural diseases and the respiratory functional diseases is the cause and process of lung cancer metastasis, the purpose of this invention is to combine the medicinal massager, ultrasound, laser, microwave and medication injection to form the local therapeutic equipment for restoration function, which is combined with operation, radiotherapy, local chemotherapy and radiofrequency ablation to form the overall therapy for killing the cancer cells as well as synchronous overall treatment to the primary lesion and multi-point metastatic lesions of the lung. Drug massage vibration is combined with the bed body that is for abdominal breathing exercises to do synchronous treatment, which is further integrated with cutaneous penetration, medication injection and breathe penetration to form the treatment that mainly aims at resuming the function of the respiratory system while killing the cancer cells at the same time. The function of resuming the respiratory system and the damage resistant treatment that is to kill the multipoint lesion structure of the cancer cell can be symmetrically, equivalently and mutually promoted to form an overall treatment, thus achieving the goal of curing the lung cancer metastasis.

In order to achieve the above goals, the technical proposal of this invention is: a kind of therapeutic apparatus for lung cancer metastasis, which is consisted of bed body, supporting structure, local therapeutic apparatus, massager, vibrator, medicinal cloth, adjustable-speed motor, motion bar, oxygen inhaler, breathing doser. It is combined with operational chemoradiotherapy to overall synchronization for the lung cancer metastasis; its characteristics are: the bed body is with a supporting structure at the back which is installed crossly and dispersedly with massager and vibrator; the medicine cloth covers the vibrator; the shoulders and feet of the bed body are fixed with adjustable-speed motors which are installed with motion bars; patient can lie down on the medicine clothe that covers the vibrator on the bed, and his arms and legs are connected with the motion bars; the motion bars on the adjustable-speed motor if started can push the patient's arms and legs back and forth to do abdominal respiration; local treatment apparatus is used after the bed treatment is finished.

The respiratory function and circulatory function of human body are closely interrelated. 95% of the cancer patients suffer from Qi-stagnation and blood stasis, which is reason why cancer is difficult to cure; however, the mutual promotion between recovering respiratory function and circulatory function can solve this problem. The cardiovascular system is closely connected with lymphatic vessels, and only recovering the mutual promotion of the two can help to recover the immune system function. In the treatment of lung cancer metastasis, it is of equal importance to recover respiratory function and kill the cancer cells in the primary lesion and metastatic lesion, which are two treatments with different nature. Only overall treatment integrated by the mutual promotion between the two can the treatment goal be reached. Single factor treatment that mainly aims at killing cancer cells in the existing technology cannot reach to this goal. Patients with lung cancer metastasis have already had the conditions of declined respiratory function and circulatory function as well as immune function that can cure cancer cells, so recovering the respiratory function alone which is constrained by the decline of circulatory function cannot gain a remarkable effect. Therefore, the treatment goal can only be achieved by the synchronous and mutual promotion of recovering respiratory function that uses bed body, vibrator, adjustable-speed motor, motion bar and other devices and recovering the circulatory function that uses massager, local treatment device; otherwise treatment goal cannot be reached if they work separately. Since there are situations where the remnant and scattered cancer cells can proliferate and metastasize, acupoint injection of anti-cancer drug and breathe dosing can be used at the same time to kill the cancer cells, thus ensuring the implementation of the treatment for recovery functions. This invention combines different factors with different natures, and the respiration and circulation as well as recovery function and killing cancer cells are symmetrical and mutually dependent factors, the lack of part will make the other part meaning less in treatment. Patients can do abdominal respiration through horizontal extensional movements pushed by the adjustable-speed motor and motion bars fixed on the bed body, and the vibrator can purify the lung and alleviate bronchial asthma and respiratory disease symptoms as well as supply oxygen to recover respiratory function. The massager can carry out massotheraphy on patients' waist and back, and the local therapeutic equipment can carry out circulatory function recovery massage from patients' arms and legs to heart and can also carry out power input treatment around the lymphoid organs to recover patient's immunity. Anti-cancer drug injection in the acupoint and the pain spot at the microcirculation disturbance part, breathe dosing of the atomized anti-cancer drugs can kill the cancer cells, which is necessary. The synchronization of all-around treatments should be used to reach the goal of resuming respiratory function. The combination of local therapeutic equipment that recovers functions and the localized radiotherapy that kills cancer cells can carry out injure resistant treatment of killing cancer cell structures at the several metastatic lesions of the lung cancer metastasis. The mutual promotion and synchronization of recovering respiratory function and killing cancer cells to treat the lesions can reach to the goal of obvious effects in treating the lung cancer metastasis.

In order to fully disclose this invention, there will be detailed description of relevant theories and technologies of this invention in treating lung cancer metastasis.

### I. Pathogeny and Development of Lung Cancer Metastasis

The Chinese philosopher Feng Youlan pointed out: philosophy and science reinforced each other. Science can increase people's positive knowledge, but cannot improve people's state; on the contrary, philosophy can improve people's state but cannot increase people's positive knowledge. The famous scientist Albert Einstein thought that if science is interpreted as the search for knowledge in the most common and extensive forms, then philosophy can be regarded as the origin of all sciences. According to the treatment principles and methods for serious diseases combined by philosophy and science, this invention draws the Schematic Diagram of the Generation and Development of Lung Cancer Metastasis to overall understand the lung cancer metastasis.

1. It is seen through the Schematic Diagram that the cause for lung cancer metastasis is the interaction between functional lesion and structural lesion of the sublevel parts of the body systems. The mutual promotion between recovering the sublevel functions of the respiratory system (including relevant blood circulation and lymphoid immunity, etc.) and the anti-injure treatment to the sublevel functions of the respiratory system (including the anti-injure treatment to relevant blood circulation and lymphoid immunity, etc.), is the treatment to the cause of lung cancer metastasis.

2. It is seen through the Schematic Diagram that human body function also has sublevel parts as the same as human body structure, and the structural diseased parts and the functional diseased parts of the lung cancer metastasis are all organ level and system level; if there is no general functional lesion that needs salvage or even the deadly general structural lesion, and the systemic chemotherapy cannot be used if it's not general lesion, which has large side effects while little curative effects, then the treatment aiming at the diseased parts will be given up. The modern technology still knows nothing about the functional diseased parts, thus there is a lack of treatment that aims at the diseased parts.

3. It is seen through the Schematic Diagram that each process and stage of the lung cancer development is the process and result of the interaction between structure and function. If function is normal, the structural lesion will be limited from development, and the patient won't die even though he is seriously ill. This is why a quarter of the aged around 80 years old have tumor in their bodies during autopsy, but there is no symptom related to cancer before their death which is caused by other diseases or reasons. In addition, it also indicates that lacking of targeted function treatment is the direct reason for the death of patients with cancer in modern technology.

4. It is also seen in the Schematic Diagram that in the interaction between same levels, it is structure that decides the gradual change and quantitative change of the self-improvement of function, while in the interaction between different levels, it is the symmetrical and equivalent combination of structure and function that decides the mutation and qualitative change processes. The existing technology still adopts the way of structure deciding function of the gradual change process in the mutation process. Treatment that uses surgery radiotherapy and chemotherapy to kill cancer cells still belongs to local treatment and won't have decisive effects, no matter how perfect or improved. What's more, the existing comprehensive treatments such as surgery combined with radiation and chemotherapy as well as radiotherapy combined with chemotherapy are the treatment combinations with the same nature as killing cancer cells, which is a way of quantity increase and can improve forces but won't generate qualitative change. Only the union combined by the two factors with different natures: functional recovery and killing cancer cells can play a decisive role.

### II. Treatment Principles and Methods about the Lung Cancer Metastasis

Similarly, according to the treatment principles and methods for serious diseases, this invention draws the Schematic Diagram of the Treatment Principles and Methods for Lung Cancer Metastasis. Overall treatment plan should be made and the advanced treatment technology combining physics and chemistry should be used to treat lung cancer metastasis. Since that the patients have already had the structural damage and decrease of function as well as the states of physical lesions and chemical lesions in their body, the injury resistant and function recovery treatment as well as physical therapy and chemical therapy are symmetrical in natures and are closed dependent to each other, though their therapeutic effects are different from person to person, and their separation will cause failure. The overall therapy and advanced therapy are described as follows:

1. The Function Recovery Treatment and its Treatment Technology in the Overall Therapy

The function recovery treatment of the therapeutic apparatus for lung cancer metastasis refers to the treatment that adopts advanced treatment technology combining the physics and chemistry and mainly aims to resume the function of the respiratory system and relevant circulatory functions and functions of immune system, while killing the cancer cells through structural injury resistance.

Treatment Mainly Aims at Resuming the Function of the Respiratory System

The abdominal respiration function that takes three quarters of the total breathing volume will be lost due to overmuch vertical movements of the human body, and then cause insufficient blood flow volume, oxygen uptake in the lung and make the lung cells in long-term idle and even inactive, which is the reason why the respiratory function declines and diseases are difficult to cure. The blood pressure added by gravity makes the blood vessel under the heart swelling, and about 500 ml to 600 ml of blood will be delayed which will make the total volume of the blood circulation reduce 15%; then arms and legs as well as muscle of back that take up 70% of blood capillary in human body will be stiffness, and there will be problems for the blood return under the heart, thus causing insufficient blood supply in the brain; the artery blood vessel is forced to contract regularly to enhance blood pressure which will cause hypertension. In addition, the function of the immune system that is closely related to the blood vascular system and respiratory system will decrease. Medical research shows that about 90% of illness in human body is related to immune system disorder.

This invention adopts the bed body that is fixed with adjustable-speed motor, motion bar, massage and vibrator as well as oxygen uptake and breathe dosing device, and allows patients to do horizontal extensional movements and back treatment of medicine massage without self-energy consumption. Meanwhile, the direct oxygen supply can recover patient's abdominal respiration to enlarge his vital capacity, thus increasing 3 to 5 times of the gas transfer velocity and improving arterial oxygen tension; internal massage on the inactive lung lobe cells can promote blood circulation and remove blood stasis, and thus raise the utilization ratio of antibiotic drugs, remove the abdominal blood stasis as well as gravity obstacles, and eliminate high blood pressure. The therapeutic massage on back and the therapeutic massage from arms and legs to heart by the local massager can increase the number of open blood capillary per cu. mm. from 31 to 1400, quicken the lymph flow to 7 times and obviously relieve the cardiac load. Back vibration device can relax the stiff muscles, and increase 10 to 16 times of the blood flow rate; in addition, it can alleviate the bronchial asthma and others suffered by the patients with lung cancers. Patients can lie down on the bed and receive around 10 minutes of thorax with its vibration frequency at 30 to 45 HZ, and generally lung can be purified and symptoms can be remarkably alleviated after several courses of treatment. At the same time of recovering cardiopulmonary function, local therapeutic equipment is used to input mechanical energy, heat and chemical energy in the lymphoid organs of the immune system and the immunocompetent cells, in order to carry out treatment for recovering immune function. Generally, there will be no reactions of advanced lung cancer and lung cancer metastasis on immunotherapy and recovering immune function. However, there will be remarkable treatment effects to carry out treatment for recovering immune function on the basis of recovering cardiopulmonary function, because cardiopulmonary function is closely related to immune function.

Complementary Therapy for Respiratory System Structure Anti-injury

Because there have already existed a small amount of cancer cells in the respiratory system of patients with lung cancer metastasis and the proliferation of cancer cells will be promoted at the same time of recovering respiratory function, it is very necessary to carry out cancer cell killing treatment while recovering the respiratory function and immune function to inhibit cancer cells. Mainly the following therapic methods are adopted: acupoint injection related to cancer, injection on microcirculation disturbance place of tender point, intramuscular injection, indirect lymphatic subcutaneous injection (but the injection site makes the drugs not go into the blood vessels but enter the lymphatic system) and breathing administration. If the diseases lie in viscera, the Back-shu point shall be pressed, with supplement of massaging the He-sea acupoint. For lung cancer patients, take Feishu point, He-sea acupoint, Chize acupoint and Feire acupoint (‘T’3-4 Jiaji acupuncture). If lung cancer patients have cough, gasp, dyspnea and retrosternal pain, Zhongchuan acupoint shall be used (5 points by T5-6, namely Ganre acupoint). As for people with insufficient lung capacity, acupoints like Sanli acupoint, Dashu acupoint and Feishu point shall be pressed. Breathing administration makes anti-cancer drugs to be produced as aerosol. At present, only 20% to 50% of the rated dose of super living aerosol can enter the lungs and all of the rest of it are impeded from the palate of oral cavity. However, in horizontal abdominal breathing, the phenomenon that the patients might breathe heavily, which can get this circumstance improved.

In acupoint anti-cancer injection solution, there are the following ways:

### (1) Acupoint Injection No. 1

[symbol] Side medicine composition [symbol] Berberine injection. [symbol] Acupoint selection [symbol] Fengmen point, Dazhu point, Dazhui point & Feishu point.

[symbol] Function [symbol] Detoxification & dissolving lumps. [symbol] Major function [symbol] Lung cancer

[symbol] Usage [symbol] 4 ml each time, once per day. 10 times for a period of treatment and rest for 3 days within therapeutic courses [symbol] Origin [symbol] Experiential Recipes

### (2) Acupoint Injection No. 2

[symbol] Side medicine composition [symbol] Houttuynia cordata injection. [symbol] Acupoint selection [symbol] Tiantu point.

[symbol] Function [symbol] Detoxification, detumescence & dissolving lumps. [symbol] Major function [symbol] Lung cancer

[symbol] Usage [symbol] Get the 5 ml injector with No. 6 or No. 7 syringe needle filled with 2 ml Houttuynia cordata injection onto Tiantu point for injection. Firstly stab for two points, then move it quickly for 1 to 2 inches down along the posterior border of bone with the angle of 30° to make the patients swallow. If the patients feel like fishbone infarction in the throat, the drug can be slowly pushed in for 1-2 ml. 2 times a day & 7 days for a course of treatment. [symbol] Origin [symbol] Experiential Recipes

In commonly used Chinese herbal medicine injection, the muscular injection includes Lianbo injection, Venenum Bufonis injection (toad venom injection), polyporus polysaccharide injection and aconite injection. In acupoint injection, there are types of Oldenlandia diffusa injection, compound angelica injection and Acanthopanax senticosus injection. The chemotherapy drugs used for acupoint injection include: Thiophosphoramidc (TSPA), Methotrexate (MTX) and AT1258. Injection dosage: 2-4 ml each time and respectively fill it into 2 to 4 acupoints. The injection method is the same as that of the general acupoint injection and it is advisable to be injected two or three times per week. In the case that the acupoint injection of chemotherapy drugs is adopted at the same time, Chinese herbal preparations can be used as solvent and shall be used according to the above method after being fully dissolved. Chinese herbal medicine injection can be used for a long time, without limitation of white blood cells and medicine doses. The chemotherapy drugs should be used in strict accordance with the amount of drug treatment and after the treatment course ends, the use of them can be stopped for 1 to 2 weeks.

At the same time, transdermal drug delivery technology can be applied to conduct the recovery function and to kill cancer cells. This invention adopts local therapeutic apparatus for high-speed extrusion & friction and transdermal drug delivery, but many macromolecular drugs including Chinese medicine are very difficult to go through the stratum corneum, which leads to small transdermal absorption and little amount of drug delivery. It mainly relies on the increase of transdermal drug delivery area. If you need to increase the drug delivery dosing on smaller area and adopt macromolecular drugs and traditional Chinese medicine (TCM), you can apply the series of products by this invention. In existing technologies, there is the product with patent number ZL201220539707.6 in Uremia Skin Dialysis Therapeutic Device. This technology adopts extrusion, friction and transdermal drug delivery in combination with ultrasound transdermal drug delivery. Here the ultrasonic empty effect can get macromolecular drugs go through the stratum corneum, improve microcirculation and input thermal energy by extrusion and friction and promote the absorption of drugs. In such a way, the above-mentioned problems can be solved.

Hereinafter several transdermal absorption drugs will be introduced:
(1) Carmustine [symbol] Effect & Purpose [symbol] Anticancer with broad spectrum. It works fast and can pass through blood-brain barrier. It has good curative effect on the treatment of brain and spinal metastasis and has certain effect on lung cancer and lymphoma. [symbol] Usage & Dosage [symbol] Take 10 ml of this drug and dissolve it into 95% ethanol. Then add water to thin up to 30 ml and do drug massage for once/d. Insist on this method for 1 to 4 months.
(2) Glyciphosphoramide [symbol] Effect & Purpose [symbol] Clinically it is mainly used for the treatment of small-cell lung cancer (SCLC), breast cancer and malignant lymphoma and the curative effect is very good. [symbol] Usage & Dosage [symbol] Local application of 20% Glyfosfinum dimethyl sulfoxide solution for 2 times/d. It can be continuously used for 20-30 days and it has fine effect on cancer, turgescence and ulcer.

2. Structural Anti-injury Treatment & Therapeutic Technologies in Whole Treatment

The structural anti-injury treatment is divided into two parts: the primary treatment of structural anti-injury treatment and the auxiliary treatment of recovery function

\textcircled{1} The Primary Treatment of Respiratory System Structure Anti-injury

The system structural injury of lung cancer metastasis refers to the multiple diseases in which the original cancer lesions are transferred from lungs to other body areas like brain, adrenal gland, liver, lungs and bone. The systemic chemotherapy including chemotherapy and molecular targeted therapy is the basic treatment means for these patients and its main goal is to alleviate symptoms, maintain the quality of life and prolong the life. The author of Lung Cancer Metastasis explicitly pointed out on the first page of the first chapter of “overview of lung cancer”: the patients with advanced lung cancer metastasis are incurable and the chemotherapy can only relieve symptoms and elongate life cycle. Therefore the modern medicine adopts operation radiation and chemotherapy and uses the respiratory system structural damage as the primary treatment. Just as seen in Lung Cancer Metastasis Therapy Principle & Technical Schematic, it is just a part of the overall treatment and it cannot achieve the cure goal. The current clinical chemotherapy belongs to intravenous systemic medication and more than 95% drugs are distributed in the normal tissues of the whole body. Only less than 5% of the drugs act on the tumor and instead the lethality of chemotherapy drugs on tumor cells is not more powerful than the lesion of normal tissues. But the dispersed cancer cells in the whole body can be eliminated by the recovery function and the recovery of immune function. Thus the drug delivery method should be changed and the interventional drug delivery in local chemotherapy should be adopted to directly send the drugs around the lesions or inject them into the tumor. In such way it not only can improve the curative effect by aiming at local drug delivery, but also can create conditions for the integration with the local therapeutic apparatus in this invention as a whole. According to the existing clinical repots, this drug delivery method is applicable to dozens of cancers such as lung cancer and breast cancer.

\hat{2} The Auxiliary Treatment of Respiratory System Structural Recovery Function

According to the description in Lung Cancer Metastasis: “More and more evidences show that the immune cells in tumor microenvironment can play a key role in the process of the proliferation and spread of tumor. The physical state is a major determinant of the prognosis on the advanced lung cancer patients. To adjust the metastasis phenotypes by intervening the host's natural immune response is a promising field in future tumor treatment.”

At the same time, it can be seen from Lung Cancer Metastasis Therapy Principle & Technical Schematic that to combine the complementary treatment of metabolic function recovery with the existing technologies of primary cancer cell killing structural anti-injury treatment, to make them become the treatment as a whole and to locate the treatment of metastasis lesions can all intensity the cancer cell lethality, reduce side effects and achieve the goal of controlling and eliminating the metastasis lesions.

The local therapeutic apparatus in this invention is just the Cancer Spread & Metastasis Therapeutic Apparatus in existing technologies. It is made of multifunctional friction head, laser, ultrasonic, microwave, radio frequency (rf), drug injection and intervention. It can input the mechanical energy, thermal energy and chemical energy into the cancer spread &metastasis diseased region of the patients, can do therapy that is mainly based on recovery function and auxiliarily depends on killing cancer cells and can be symmetric and equivalent with the primary treatment of cancer cell killing structural anti-injury in surgery radiation & chemotherapy to mutually promote the combination into a whole and cure cancer spread & metastasis lesions. Its application has been explained in detail in Cancer Spread & Metastasis Therapeutic Apparatus. Here is a brief introduction about it: For the existing technologies, before the operation, the preoperative radiotherapy and preoperational chemotherapy must be done. However, the local therapeutic apparatus takes the lead in the radiotherapy of circle center site for 5 minutes of treatment. And then it starts the radiation therapy and consequently eliminates “differential concentric circles effect” which has bigger side effects to make the radiotherapy go on the rails. For local chemotherapy, the local therapeutic apparatus carries out 5 minutes of treatment on the lesion sites after drug injection or being input tumor lesions for several minutes. The commonly used drugs for interventional chemotherapy are still cisplatin, mitomycin and epirubicin, etc. Because radiotherapy can hurt the normal tissues and result in the unwilling termination of tumor radiotherapy of patients, at the time of radiotherapy, for maximum protection of the normal tissue cells and killing of cancer cells to the greatest extent, the local therapeutic apparatus is needed to adopted for treatment. But the local chemotherapy concentrates chemotherapeutic drugs around and inside the tumor lesions and it also needs to apply the local therapeutic apparatus to promote the absorption and use of drugs because the surface of tumor cells can be changed in the face of anti-cancer drugs, leading to the reduction of intake and increase of discharge. Accordingly the drug concentration is not enough, resulting in the decline of curative effect and even the occurrence of no effect or drug resistance. And the superficial changes are caused by the insufficiency of mechanical energy and physical energy. The effect of single thermal energy input is limited and it needs to input mechanical energy and thermal energy for mutual promotion, which can change this situation and promote the absorption and utilization of drugs. At present, the variety of all kinds of patented chemical anti-cancer drugs in the world can reach more than one thousand and there is no acknowledged anti-cancer drug with special effect. The drug resistance of cancers leads to the failure of 90% of metastatic cancer carcinoma treatment. Various kinds of radiotherapy equipment like radio frequency, ultrasonic gathering, microwave, radioactive cobalt, Gamma knife and intensity modulated radiation can enlarge the treatment scope, reduce side effects and do palliative care. However, it has little effect on the improvement of cancer cure rate. Therefore, the cure of cancer must adopt etiological treatment in which the structure and function can be promoted by each other and use the advanced treatment technologies in which physics and chemistry can be mutually promoted. In radiofrequency ablation treatment of metastatic lesions, in the treatment of lymphatic metastatic lesions & recovery of the function around lesions and in the treatment of hematogenous metastasis lesions & recovery of function around lesions, the local therapeutic apparatus should be combined with radiotherapy and local chemotherapy. This invention adopts anti-cancer Chinese herbal medicine injection, chemical drug acupoint injection and microcirculation barrier tender point injection on the lesions and sites around the lesions and uses the combined treatment with mechanical energy, ultrasonic vibration energy and laser heat energy to be transported and massaged by local therapeutic apparatus. No matter how much the surgery radiotherapy & chemotherapy, ultrasonic and laser in anti-cancer treatment technologies to be perfected and improved, either of them has the nature of killing cancer cells and the therapy belongs to specific therapy and is able to treat the single metastatic lesions of few lung cancer patients and to lengthen life. This invention combines the treatment of recovery function with general significance with specific therapy, which could produce the overall qualitative changes. It can cure multiple metastasis lesions and can make prolong the life of most of lung cancer metastasis patients.

### III. On Structure & Function of Lung Cancer Metastasis Therapeutic Apparatus

This invention is an integration composed by the symmetry of local therapeutic apparatus and bed body curer. Its features are as follows: two parts mutually depend on each other and act synchronically on each other and they do the treatment of function recovery through the overall acting force generated by the mutual promotion of each other. At the same time, it also has the structural anti-injury curative effect on killing cancer cells. In the treatment of lung cancer metastasis, it should be symmetrically combined with surgery radiotherapy & chemotherapy and should act synchronically with it to become an overall part for treatment. They cannot be separated for independent application.

The Chinese medicine insists that Qi and blood are two kinds of basic matter constituting the human body. Qi is closely related to blood, thus there are the sayings of “qi acts the master of blood” and “Blood is fluent if Qi is fluent; and blood gets blocked if Qi is in stasis”. In all the microcirculation material exchanges in human body, the matter filtered and absorbed by its blood capillary all belong to liquid solution and only the matter filtered and absorbed by pulmonary capillary vessels is gas. Breathing is the dominant link for pulmonary microcirculation and 95% of patients with advanced lung cancer have the diseased Qi stagnation and blood stasis at the same time.

In the massage & vibration respiration movement bed body of this invention, the electromagnetic vibrator and motor vibrator in both massager and vibrator belong to technical products. The products have the uniform model and size and they are arranged and installed at the middle lower back of the bed body in reciprocal chiasma order and the stypes with drug juice that can activate blood circulation are applied on them. There are totally four speed regulating motors in the shoulder and leg parts at both sides of the bed body. The movable rod is fixedly installed with the speed regulating motor together and at the head of bed body, oxygen supply device and respiratory drug delivery apparatus are installed. Patients lie on the bed body and below the lower back are stypes. The friction head in massager under the stypes rotates and does drug massage towards the lower back of human body through these stypes to promote blood circulation. The vibrators relax the muscles by means of vibration and massage through stypes, get the lungs purified and improve the respiratory disease symptoms. The limbs of patient are connected and fixed with the movable rod while the speed regulating motor does round-trip rotational movement and drives the movable rod and the limbs of patients to do horizontal stretching back and forth. The above-mentioned process is abdominal breathing movement. The activation of oxygen inhalator at the same time can make patients get more oxygen supply and improve the respiratory function. The critically ill patients can receive the respiratory drug delivery treatment at the same time. After the bed body treatment ends, the local therapeutic apparatus should be utilized to do limb centripetal medicine massage treatment on patients so as to improve the heart function.

While recovering the respiratory system function and related circulation function as well as immune function, the acupoint injection, injection on tender point microcirculation barrier and injection on easily transferrable sites should be done for patients. Drugs include Chinese herbal anti-cancer drugs and chemical anti-cancer drugs. Drug injection can avoid the spread of cancer cells while the treatment of recovery function is independently done. The recovery of respiratory function and promotion of circulation function can mutually help each other and can act on each other synchronously. Moreover, the immune function can be restored. The elimination of production & metastasis environment of cancer cells inside the human body can be realized through the recovery function and the combination with fixed-point elimination of metastatic lesion treatment can form the overall treatment, generate the qualitative change and attain the goal of controlling and eliminating lung cancer metastasis for treatment. In therapy, other configurations can also achieve the purpose of this invention.

### IV. Comparison with Existing Technology

1. From generation and development sketch map of lung cancer metastasis, we can see that advanced lung cancer results from interaction between respiratory organ structural lesion and respiratory system functional lesion, which is bulk property lesion. Under existing technology, we mostly apply systemic chemotherapy and molecular targeted drug to process palliative treatment; operation, radiotherapy and other local treatment methods also can be palliative applied, whose target is relieving symptom of metastasis.

Pathogenesis diseased region is of the same importance with symptom. In this invention, massage breathing bed can therapy functional decrease of respiratory system and relevant circulatory function decrease, and immunity function decrease, and forms to overall therapy with operation, radiotherapy, local chemotherapy by mutual promotion synchronization effect with therapy organ focus, so as to control lung cancer lesion. Existing technology lacks of function therapy for recovering respiratory system, and cannot prevent generation and development of lung cancer metastasis.

2. From therapy principle and technology sketch map of lung cancer metastasis, we can see that in advanced lung cancer, organ same level lesion, as quantity change method, means that structure decides function, because the structure level of organ is higher than function level of organ, but bulk property of symmetry dependency of two side haven't changed. In fixed point lung cancer metastasis therapy, fixed therapy method of existing technology includes excision, external radiotherapy, stereotactic radiotherapy surgery, stereotactic radiotherapy therapy, and radiofrequency ablation all belong to structural resist-injury therapy for killing cancer cell. Because it is lack of fixed point recovery function, radiotherapy has “difference concentric circles effect”, which won't product entirety action and result in failure of fixed point therapy and cannot be processed in long term constantly. In existing technologies, biological therapy and traditional Chinese medicine therapy are adjuvant therapy suitable to the case matching to operation, radiotherapy, chemo-radiotherapy, and it cannot have synchronization therapy effect at some position with operation, radiotherapy, chemo-radiotherapy and lack of symmetry dependency, and mutual combination can result in quantity variation but not feature variation. In overall therapy, it lacks of symmetry party, the existence significance of the other party is restricted. This invention applies fixed point recovery function of local therapy equipment, which has interaction effect at same position with fixed point operation radiotherapy, local chemotherapy, and radiofrequency ablation, and it forms overall therapy and produces feature variation so that it can process therapy on metastatic lesion for most lung cancer metastasis patients to get the target of controlling and remove focus and extending life wo most lung cancer patients.

### V. SUMMARY

Scientific researches show that: internal human body has huge anticancer ability, so if it is properly mobilized and developed, the anticancer ability of human will be tens of times than normal situation, moreover, inner regulation f human immunologic balance doesn't have side effect. Many medical scientists clearly presented that the first principle of therapeutic is the application of spontaneous recovery. Metabolism function is the base of human life, but existing technology is not ideal by applying operation and various specificity therapeutic effects in curing cancer and other serious diseases, one of the biggest problem is that they haven't designed recovery mechanism and ability of human into the whole therapy plan, and don't have the measures of relying, mobilizing and exerting its effect. Theoretically, they admit the self-healing of human, but they don't have specific position in therapy principle.

According to natural law, material metabolism, energy metabolism, structure inner cause, and function outer cause are symmetry and equivalent. They are interdepend and of same importance, lack of equal recovery function therapy by cancer patients is the direct reason for failure of cancer therapy. In order to cure cancer, this invention drew generation and development sketch map of lung cancer metastasis and therapy principle and technology sketch map of lung cancer metastasis based on combination of philosophy and science, and expects to put effort on innovating therapy principle and technology for curing lung cancer metastasis, and give living hope to patients who cannot be cured currently.

### BRIEF INTRODUCTION OF FIGURES

FIG. 1. Front view of lung cancer metastasis therapy equipment

FIGS. 2(A) and 2(B). Supporting structure, massager, vibrator of therapy equipment

FIG. 3. Local therapy equipment in therapy equipment showed by FIG. 1

FIG. 4. Generation and development sketch map of lung cancer metastasis

FIG. 5. Therapy principle and technology for curing lung cancer metastasis

### Specific Execution Mode

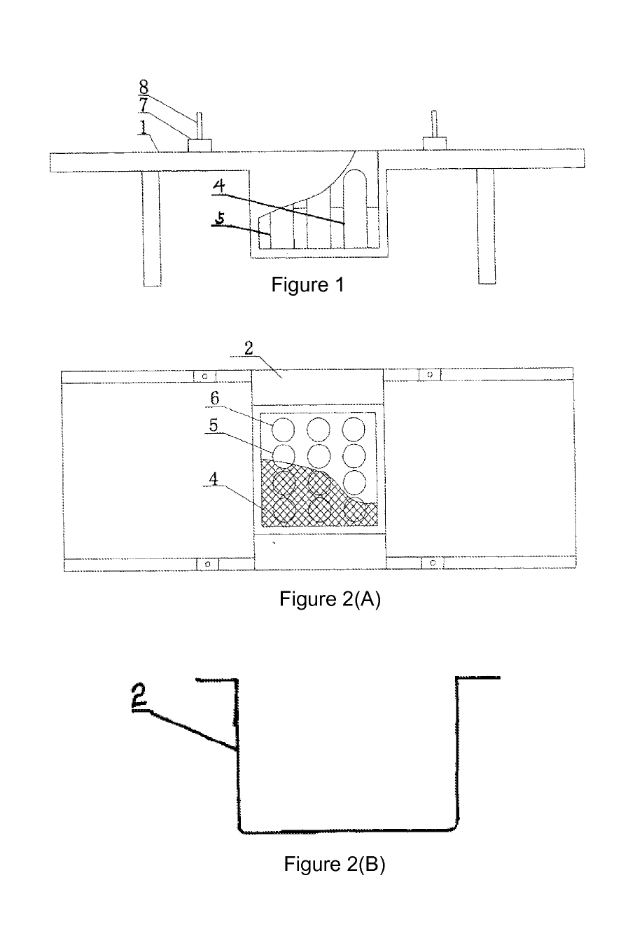

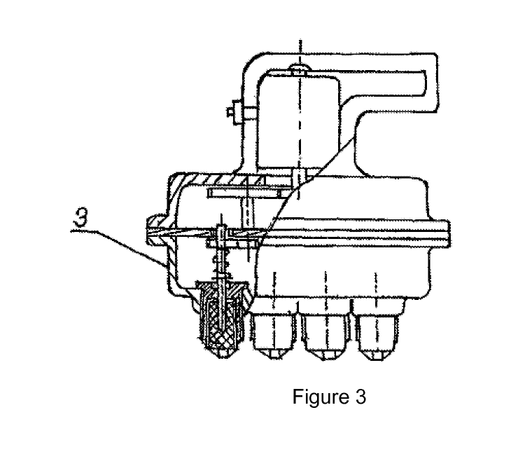

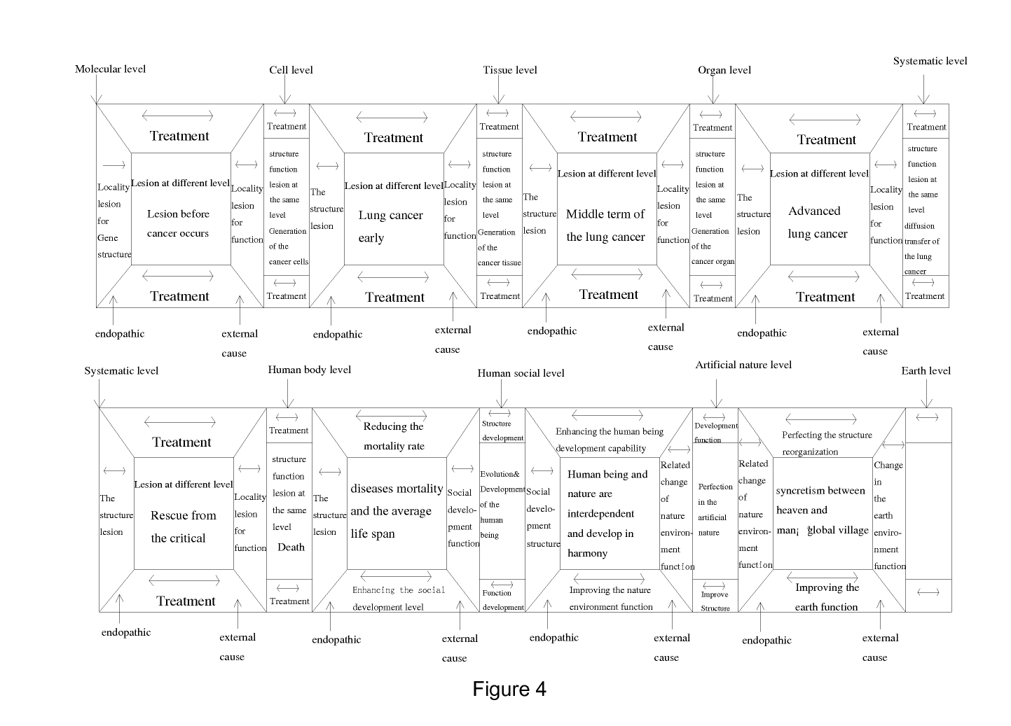

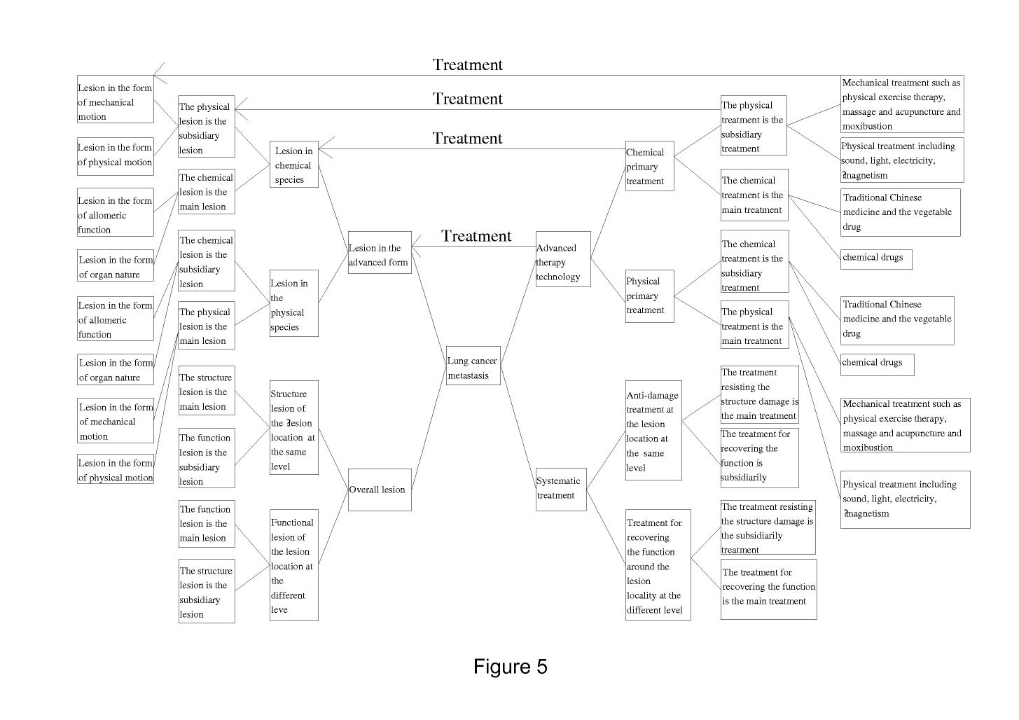

In the lung cancer metastasis therapy equipment of this invention showed in FIG. 1: 1 is bed, 2 is supporting structure, 3 is local therapy equipment, 4 is massager, 5 is vibrator, 6 is stype, 7 is adjustable-speed motor, 8 is motion bar. FIGS. 2 and 3 is supporting structure, massager, vibrator and local therapy equipment in therapy equipment showed in FIG. 1.

The therapy equipment of this invention is achieved by following technical measure: a lung cancer metastasis therapy equipment is consisted of bed, supporting structure, local therapy equipment, massager, vibrator, stype, adjustable-speed motor, motion bar, oxygen inhaler, breath doser, and have comprehensive synchronization effect with operation radiotherapy and chemotherapy to cure cancer metastasis. Its features are: the wrist part of bed 1 has supporting structure 2, cross dispersion of supporting structure 2 is installed with massager 4, vibrator 5, massager 4, vibrator 5 is covered with stype 6, shoulders of two sides and leg at bed 1 is installed with adjustable-speed motor 7, adjustable-speed motor 7 is installed with motion bar 8, and the patient lies on massager 4, vibrator 5 and stype 6 in bed 1, arms and legs are connected with motion bar 8, start motion bar 8 and adjustable-speed motor 7 to drive arms and legs of patients to have to-and fro movement and abdominal respiration, local therapy equipment 3 therapy is processed when the therapy on be 1 is completed.

The working principle of this invention is:

1. Massager and vibrator are equipped with micro motor, which is existing technical product equipped in intensive cross dispersion, whose number is 6 to 50 and the model and size are the same. It can process therapy after starting micro-motor, and the combination of massager and stype with soup can form medicine massage, so the soup can make breathing function recovery and blood circulation recovery medicine according to requirement of patients. While massage vibration therapy, starting adjustable-speed motor to drive motion bar to have to-and-fro rotation so as to drive arms and legs of patient to have horizontal stretching exercise and produce abdominal respiration. It can process viscera massage recovery breath function through up and down movement of diaphragm, and at the same time oxygen uptake can enlarge intensity of metabolism. Acupoint injection of medication, breath dose, medicine massage of arms and leg by local therapy equipment can be process in the same time with medicine massage around lymphoid organ. The characteristic is that the synchronization of different therapies cannot be separated, otherwise, it will cause failure, because structural function, physical form and chemical species rely on each other and cannot be separated.

2. In lung cancer metastasis and fixed point therapy on original lesion and metastatic lesions, all the local cancer cell killing therapy methods: operation, radiotherapy, local chemotherapy, radiofrequency ablation must be symmetry with local therapy in this invention to form entire synchronization, whose target is reducing side effect and enlarge cancer-killing force and recovery function without local therapy cannot achieve target.

3. The target of recovering breathing system is removing surrounding environment factor of producing cancer cells; in the early and medium stage, this invention can process the therapy with primary target of recovering function, and with assisting stage of killing cancer cell alone after radiotherapy and chemotherapy, because this method belongs to lung cancer quantitative change process, so human body itself has the ability of changing disease development with the coordination of outer effect. In advanced stage of lung cancer and metastasis, it belongs to qualitative change and mutation stage, and has direct threat to human life. At present, conventional radiotherapy, stereotactic radiosurgery, stereotactic radiotherapy, radiofrequency ablation and other minimally invasive surgical technologies still has high local control efficiency, however, most patients who accepted fixed point therapy dead for whole body development of disease, but not local destruction, so only when local part and whole body, inner structure therapy and external functional therapy react with each other can produces feature change, it can convert patient's condition development. This invention has to have joint therapy with operation radiotherapy and chemotherapy, which cannot be separated, otherwise failure will be caused, and structural function dual standard inspection in therapy process.

The specific steps for therapy are as follows:

1. According to specific method explained above, this invention is applied to have recovery therapy on breath system's function, the therapy factors of belonged parts will be process together, and the therapy time is 8 minutes. The temperature of ultrasonic wave, laser, microwave and other local heating equipment in therapy equipment is 42 to 50°.

2. 3 minutes after recovery function therapy, operation and radiotherapy can be processed. After intervention dose of local chemotherapy, intervention injection dose, and local therapy equipment will be applied for 5 minutes.

3. If the effect force of operation, radiotherapy, and chemotherapy is the process is reduced, the times for therapies can be increased for every next day, and 15 times for a treatment course. In a word, if treatment mode is affirmed, the therapy method and times are confirmed according to patient condition and constitution. Structural and functional dual standard inspection will be processed in and after therapy procedure

### EXAMPLE FOR THE IMPLEMENTATION OF THIS INVENTION

Massage and local therapy in bed of this invention are applied to process centrality medicine massage and to cancer patients. It is “massager therapy equipment” national invention patented product, ZL98110305.7, which is approved by Food and Drug Administration of Shandong Province, and got Shandong medicine administrative equipment (test) 2000 No. 2020044. It is applied at Qingdao hospital, Qingdao Chinese medicine hospital, Qingdao tumor hospital, the third people's hospital of Qingdao for many year, and the clinical effect is obvious, which is welcome by patients. Horizontal stretching exercise drives abdominal respiration, which can improve breathing function and oxygen uptake, so it is widely applied in clinical rehabilitation, and is proved to be effective.

1. A lung cancer metastasis therapeutic equipment, consisting of a lathe bed, a supporting structure, a local therapeutic equipment, a massager, a vibrator, a stype, an adjustable-speed motor, a motion bar, oxygen inhaler, and a drug delivery device via breathing, wherein, the supporting structure is arranged in a waist back of the lathe bed, the massager and the vibrator are arranged on the supporting structure crossly and dispersedly respectively and are covered with the stype, the adjustable-speed motor is arranged at a location of shoulders and legs of a patient in two sides of the lathe bed, and the motion bar is fixed on the adjustable-speed motor.

2. An application method of the lung cancer metastasis therapeutic equipment according to claim 1, comprising, lying a patient on the stype covered on the massager and the vibrator, whose arms and legs are connected with the motion bar, and initiating the adjustable-speed motor, thereby the motion bar leads back and forth movement of arms and legs of the patient to accomplish abdominal respiration.

3. The application method of the lung cancer metastasis therapeutic equipment according to claim 2, comprises the following steps:
performing synchronization treatment for 8 minutes to recover function of a respiratory system of the patient; a heating temperature of local heating devices by means of ultrasonic, laser, or microwave, being between 42 and 50° C.;
three minutes after the treatment for recovering the function, performing surgery and chemoradiotherapy, wherein, the massotheraphy with injections in the whole body for 5 minutes is performed by means of the local therapeutic equipment after interventional administration and administration of intervention injection, and a systemic massotheraphy by means of the local therapeutic equipment after systemic chemotherapy;
wherein, action intensity of the surgery and the chemoradiotherapy is decreased and of which the treatment frequency may is increased to once every other day, and each course of treatment takes 15 days.

4. (canceled)

5. The lung cancer metastasis therapeutic equipment according to claim 1, wherein six to fifty micromotors are installed fixedly on both of the massager and the vibrator.

6. The application method of the lung cancer metastasis therapeutic equipment according to claim 2, wherein the motion bar leads horizontal extensional movements of the arms and legs of the patient to accomplish the abdominal respiration.

7. The application method of the lung cancer metastasis therapeutic equipment according to claim 3, wherein the stype is coated with a liquid medicine for recovering the respiratory function and blood circulation.

8. The application method of the lung cancer metastasis therapeutic equipment according to claim 3, wherein the medicine injection pointing to the acupoint, the administration via respiration, the massotheraphy with injections in the arms and legs and around the lymphoid organ via the local therapeutic equipment occur at the same time.

9. The application method of the lung cancer metastasis therapeutic equipment according to claim 3, wherein the steps are performed in synchronization and are inseparable.
